SEQUENCE LISTING (1 sequence)

SEQ ID NO: 1 - protein, 889 aa, synthetic construct
AGADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPAKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLPWGGGGSDIELTQSPSSLAVSAGEKVTMSCKSSQSILYSSNQKNYLAWYQQKPGQSPKLLIYWASTRESGVPDRFTGSGSGTDFTLTISSVQAEDLAVYYCHQYLSSYTFGGGTKLEIKGGGGSQVQLQQSGPELVRPGASVRISCKASGYTFASYYIQWMKQRPGQGLEWIGWINPGNVNTKYNEKFKGKATLTADKSSTTAYMQLSSLTSEDSAVYFCARSTYYRPLDYWGQGTTVTVSSGGGGSGGGGSGGGGSDIELTQSPSSLAVSAGEKVTMSCKSSQSILYSSNQKNYLAWYQQKPGQSPKLLIYWASTRESGVPDRFTGSGSGTDFTLTISSVQAEDLAVYYCHQYLSSYTFGGGTKLEIKGGGGSQVQLQQSGPELVRPGASVRISCKASGYTFASYYIQWMKQRPGQGLEWIGWINPGNVNTKYNEKFKGKATLTADKSSTTAYMQLSSLTSEDSAVYFCARSTYYRPLDYWGQGTTVTVSSHHHHHH

(19) United States
(12) Patent Application Publication
(10) Pub. No.: US 2026/0137777 A1
(43) Pub. Date: May 21, 2026

(54) IMMUNOTOXIN FOR CANCER IMMUNOTHERAPY

(71) Applicant: The Regents of the University of Colorado, a Body Corporate, Denver, CO (US)

(72) Inventors: Zhirui Wang, Denver, CO (US); Jihong Ma, Denver, CO (US); Zhaohui Wang, Denver, CO (US)

(21) Appl. No.: 19/131,513

(22) PCT Filed: Nov. 21, 2023
(86) PCT No.: PCT/US2023/080722

§ 371 Date: May 20, 2025

Related U.S. Application Data
(60) Provisional application No. 63/384,562, filed on Nov. 21, 2022.
(60) Provisional application No. 63/384,601, filed on Nov. 22, 2022.

(51) Int. Cl. A61K 39/395; A61K 38/16; A61K 39/00; A61P 35/00; C07K 16/28

(52) U.S. Cl. CPC A61K 39/3955; A61K 38/164; A61P 35/00; C07K 16/2818; C07K 16/2866; A61K 2039/507; C07K 2317/626; C07K 2319/55

(57) ABSTRACT

Methods of treating various types of cancer, including breast cancer, involve administering a therapeutically effective amount of a pharmaceutical composition containing a genetically engineered C-C Motif Chemokine Receptor 4 immunotoxin alone or in combination with one or more additional therapeutic agents, such as a pharmaceutical composition containing an anti-programmed death-1 monoclonal antibody.

### CROSS-REFERENCE TO RELATED APPLICATIONS

This application claims priority to U.S. Provisional Patent Application Nos. 63/384,562 and 63/384,601, both titled “IMMUNOTOXIN FOR CANCER IMMUNOTHERAPY” and filed on Nov. 21, 2022 and Nov. 22, 2022, respectively.

The contents of each application are hereby incorporated herein in their entirety.

### TECHNICAL FIELD

The present disclosure relates generally to compositions, systems, and methods for treating cancer. Specific implementations involve the delivery of a pharmaceutical composition containing an engineered immunotoxin to a subject afflicted with cancer.

### SEQUENCE LISTING

The present disclosure further incorporates by reference the Sequence Listing submitted herewith. The Sequence Listing .xml file, identified as P306827WO01, is 2,886 bytes in size and was created on Nov. 21, 2023. The Sequence Listing, electronically filed herewith, does not extend beyond the scope of the specification, and does not contain new matter.

### BACKGROUND

Cancer affects millions of people each year, causing severe symptoms and often death. Despite substantial efforts to combat all forms of the disease, effective treatments remain elusive. The diversity of factors causing, contributing, and exacerbating various cancer types makes them extremely difficult to treat. Accordingly, safe, and effective cancer treatments are needed.

### SUMMARY

This disclosure provides novel compositions and methods for treating cancer. Embodiments involve administering a therapeutically effective amount of a disclosed pharmaceutical composition to a subject afflicted with cancer. Examples of the pharmaceutical composition may include a genetically engineered C-C Motif Chemokine Receptor 4 (“CCR4”) immunotoxin, which may comprise a diphtheria toxin-based recombinant fold-back diabody anti-human CCR4 immunotoxin (“CCR4-IT”). According to such examples, CCR4-IT may comprise two tandem anti-human CCR4 scFv fused to a truncated diphtheria toxin DT390. In some embodiments, the cancer may be breast cancer, e.g., triple-negative breast cancer.

In some examples, methods may further involve administering a pharmaceutical composition containing an anti-programmed death-1 (“PD-1”) monoclonal antibody to the subject afflicted with cancer. In some examples, the pharmaceutical composition containing the anti-PD-1 monoclonal antibody may be administered concurrently with the pharmaceutical composition containing the genetically engineered CCR4 immunotoxin. In some examples, the pharmaceutical composition containing the anti-PD-I monoclonal antibody and the pharmaceutical composition containing the genetically engineered CCR4 immunotoxin may be administered separately. In some examples, the pharmaceutical composition containing the genetically engineered CCR4 immunotoxin may also contain the anti-PD-1 monoclonal antibody.

In some examples, the CCR4 immunotoxin reduces the amount of tumor-infiltrating Treg cells in the subject. In some examples, the pharmaceutical composition is administered intravenously. In some examples, the method further involves performing a biopsy on a tumor within the subject and determining that the tumor is CCR4+. In some examples, the method may not involve determining whether a tumor is CCR4+, as administration of the CCR4-IT disclosed herein may provide an effective immunotherapy characterized at least in part by the depletion of CCR4+ tumor-infiltrating effector Tregs. Effective immunotherapy may be achieved in CCR4-tumors. In some examples, administering to the subject the therapeutically effective amount of the pharmaceutical composition causes a reduction in tumor volume, weight, number, metastasis, or combinations thereof.

In accordance with embodiments of the present disclosure, a method of treating or alleviating at least one symptom of a cancer in a subject diagnosed with the cancer involves administering to the subject a therapeutically effective amount of a pharmaceutical composition comprising a genetically engineered CCR4 immunotoxin, where the cancer comprises breast cancer. T-cell acute lymphoblastic leukemia (“T-ALL”), cutaneous T-cell lymphoma (“CTCL”), lung cancer, or head and neck cancer.

In some examples, the breast cancer comprises triple-negative breast cancer. In some examples, the CCR4 immunotoxin is a diphtheria toxin-based recombinant fold-back diabody anti-human CCR4 immunotoxin. In some examples, the CCR4 immunotoxin comprises two tandem anti-human CCR4 scFv fused to a truncated diphtheria toxin DT390. In some examples, the method further involves administering a pharmaceutical composition comprising an anti-PD-1 monoclonal antibody to the subject.

In accordance with embodiments of the present disclosure, a system for treating or alleviating at least one symptom of breast cancer in a subject diagnosed with breast cancer includes an injection device configured to administer to the subject a therapeutically effective amount of a pharmaceutical composition containing a genetically engineered CCR4 immunotoxin.

In accordance with embodiments of the present disclosure, a pharmaceutical composition formulated for treating a breast cancer in a subject includes a genetically engineered CCR4 immunotoxin and one or more excipients. In some examples, the CCR4 immunotoxin is a diphtheria toxin-based recombinant fold-back diabody anti-human CCR4 immunotoxin.

This Summary is neither intended nor should it be construed as being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and the Detailed Description and no limitation as to the scope of the present disclosure is intended by either the inclusion or non-inclusion of elements, components, etc. in this Summary. Other features and advantages of the invention will be apparent from the following Detailed Description and claims.

### BRIEF DESCRIPTION OF THE DRAWINGS

The following drawings form part of the present specification and are included to further demonstrate certain embodiments of the present disclosure. Certain embodiments can be better understood by reference to one or more of these drawings in combination with the Detailed Description of certain embodiments presented herein.

FIG. 1 is a schematic representation of a genetically engineered, truncated diphtheria toxin-based recombinant single-chain fold-back diabody anti-human CCR4 immunotoxin in accordance with embodiments disclosed herein.

FIG. 2 is a graph showing the survival days of CTCL tumor-bearing mice administered the engineered CCR4 immunotoxin in accordance with embodiments disclosed herein.

FIG. 3 is an image panel showing the livers of the tumor-bearing mice represented in FIG. 2 after necropsy.

FIG. 4 is an image panel showing hematoxylin and eosin staining of liver tissue of tumor-bearing mice shown in FIG. 3.

FIG. 5 is a graph showing the survival days of T-ALL tumor-bearing mice administered the engineered CCR4 immunotoxin in accordance with embodiments disclosed herein.

FIG. 6 is a graph showing the depletion of CCR4+Foxp3+ Treg cells in the peripheral blood of two cynomolgus monkeys upon administration of the engineered CCR4 immunotoxin in accordance with embodiments disclosed herein.

FIG. 7 is a graph showing the average volume of tumors obtained from mice afflicted with triple-negative breast cancer with and without administration of the engineered CCR4 immunotoxin in accordance with embodiments disclosed herein.

FIG. 8 is an image panel showing representative tumors reflected in FIG. 7.

FIG. 9 is a graph showing the average weight of tumors obtained from the mice represented in FIG. 7.

FIG. 10 is an image panel showing the tumor metastases measured in the mice represented in FIG. 7.

FIG. 11 is an image panel showing hematoxylin and eosin staining of tumor tissue represented in FIG. 7.

FIG. 12 is a graph showing the average volume of tumors obtained from mice afflicted with triple-negative breast cancer with and without administration of the engineered CCR4 immunotoxin alone or in combination with Pembrolizumab in accordance with embodiments disclosed herein.

FIG. 13 is a graph showing the average volume of tumors represented in FIG. 12 at 22 days post-tumor-cell injection.

FIG. 14 is a graph showing the average volume of tumors represented in FIG. 12 at 28 days post-tumor-cell injection

FIG. 15 is a graph showing the average volume of tumors represented in FIG. 12 at 30 days post-tumor-cell injection

FIG. 16 is an image panel showing representative tumors reflected in FIG. 15.

FIG. 17 is a graph showing the average weight of tumors obtained from the mice represented in FIG. 16.

FIG. 18 is a graph showing the average volume of tumors obtained from immunodeficient mice afflicted with triple-negative breast cancer with and without administration of the engineered CCR4 immunotoxin alone or in combination with Pembrolizumab in accordance with embodiments disclosed herein.

FIG. 19 is an image panel showing representative tumors reflected in FIG. 18.

FIG. 20 is a graph showing the average weight of tumors obtained from the mice represented in FIG. 18.

### DEFINITIONS

Unless defined otherwise below, all technical and scientific terms used herein have the same meaning as commonly understood by one of ordinary skill in the art to which this disclosure belongs.

As used herein, the terms “treat,” “treating,” “treatment” and the like, unless otherwise indicated, can refer to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the condition or disorder. “Treatment” may refer to targeted therapy in some examples. “Treatment” may also refer to immunotherapy in some examples, which may be defined as the use of a subject's own immune system to combat cancer.

The terms “treat”. “treating” and “treatment” may further refer to eliminating, reducing, suppressing, or ameliorating, either temporarily or permanently, either partially or completely, a clinical symptom, manifestation or progression of an event, disease or condition associated with the oncological disorders and diseases described herein. As is recognized in the pertinent field, methods and drugs employed as therapies may reduce the severity of a given disease state, but need not abolish every manifestation of the disease to be regarded as useful. Similarly, a prophylactically administered treatment need not be completely effective in preventing the onset of a condition to constitute a viable prophylactic method or agent. Simply reducing the impact of a disease (for example, as disclosed herein, decreasing cancer cell growth rate, reducing tumor size, reducing tumor weight, reducing or preventing metastasis, etc. and/or reducing the number or severity of associated symptoms, or by increasing the effectiveness of another treatment, or by producing another beneficial effect), or reducing the likelihood that the disease will occur or worsen in a subject, is sufficient. One embodiment of the invention is directed to a method for determining the efficacy of treatment comprising administering to a patient therapeutic treatment in an amount, duration, and repetition sufficient to induce a sustained improvement over pre-existing conditions, or a baseline indicator that reflects the severity of the particular disorder.

“Reducing.” “reduce.” or “reduction” means decreasing the presence, severity, or duration of a cancer, a cancer symptoms, or cancerous tumors.

As used herein. “subject” means a human or other mammal. A subject can be considered in need of treatment. The disclosed compositions, methods, and systems may be effective to treat patients diagnosed with cancer or subjects at risk of developing cancer.

“Administration of” and “administering a” compound, composition, or agent should be understood to mean providing a compound, composition, or agent, a prodrug of a compound, composition, or agent, or a pharmaceutical composition as described herein. The compound, agent or composition may be provided or administered by another person to the subject (e.g., infusion) or it may be self-administered by the subject.

“Intravenous” administration refers to administering a drug (e.g., the disclosed immunotoxin and/or pharmaceutically acceptable forms thereof) into a vein of a patient, e.g., by infusion (slow therapeutic introduction into the vein).

Intraperitoneal administration or injection refers to administering a drug (e.g., the disclosed peptide and/or pharmaceutically acceptable forms thereof) into the peritoneum of a patient.

“Infusion” or “infusing” refers to the introduction of a drug-containing solution into the body through a vein for therapeutic purposes. Generally, this is achieved via an intravenous (IV) bag.

An “intravenous bag” or “IV bag” is a bag that can hold a solution which can be administered via the vein of a patient. In one embodiment, the solution is a saline solution (e.g. about 0.9% or about 0.45% NaCl). Optionally, the IV bag is formed from polyolefin or polyvinyl chloride.

By “co-administering” is meant intravenously administering two (or more) drugs during the same administration, rather than sequential infusions of the two or more drugs. Generally, this will involve combining the two (or more) drugs into the same IV bag prior to co-administration thereof.

The terms “active ingredient” or “active pharmaceutical ingredient” as used herein refer to a pharmaceutical agent, active ingredient, compound, or substance, compositions, or mixtures thereof, that provide a pharmacological, often beneficial, effect. In some embodiments, the active ingredient is CCR4-IT, which may be co-administered with an anti-PD-1 antibody.

“Pharmaceutical compositions” or “pharmaceutical formulations” are compositions that include an amount (for example, a unit dosage) of one or more of the disclosed compounds, e.g., CCR4-IT, together with one or more non-toxic pharmaceutically acceptable additives, including carriers, diluents, and/or adjuvants, and optionally other biologically active ingredients. Such pharmaceutical compositions may be prepared by standard pharmaceutical formulation techniques such as those disclosed in Remington's Pharmaceutical Sciences, Mack Publishing Co., Easton, Pa. (19th Edition).

As used herein, a “pharmaceutically acceptable excipient” or a “pharmaceutically acceptable carrier” means a pharmaceutically acceptable material, composition, or vehicle that contributes to the desired form or consistency of the pharmaceutical composition. Each excipient or carrier must be compatible with other ingredients of the pharmaceutical composition when comingled such that interactions which would substantially reduce the efficacy of the compositions of this disclosure when administered to a subject and interactions which would result in pharmaceutical compositions that are not pharmaceutically acceptable are avoided. In addition, each excipient or carrier must be of sufficiently high purity to render it pharmaceutically acceptable. Non-limiting examples of pharmaceutically acceptable carriers may include lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methylhydroxybenzoate, propylhydroxybenzoate, talc, magnesium stearate, mineral oil or the like. In addition or alternatively, a carrier may include comprise a lubricant, a wetting agent, a flavor, an emulsifier, a suspending agent, a preservative, or the like.

The agents, compounds, compositions, antibodies, etc. used in the implementations described herein are considered to be purified and/or isolated prior to use. Purified materials are typically “substantially pure,” meaning that CCR4-IT has been isolated from other components.

As used herein, the terms “identity” or “similarity” denote relationships between two or more polypeptide sequences or their underlying nucleic acid sequences, as determined by comparing the sequences. In the art, “identity” also means the degree of sequence relatedness between polypeptide or polynucleotide sequences, as determined by the match between strings of such sequences.

The term “amelioration” as used herein refers to any improvement of a disease state (for example cancer) of a patient, by the administration of one or more treatments and/or compositions, according to the present disclosure, to such patient or subject in need thereof. Such an improvement may be seen as a slowing down the progression or stopping the progression of the disease of the patient, and/or as a decrease in severity of disease symptoms, an increase in frequency or duration of disease symptom-free periods or a prevention of impairment or disability due to the disease.

An amino acid within the disclosed peptide compositions can be substituted to create an engineered CCR4-IT peptide molecule. In some embodiments, the amino acids of the disclosed peptides may be natural, synthetic, for example derivatized native or non-native amino acids, for example diethyl lysine. The amino acid residue can be replaced by a residue having similar physiochemical characteristics, that is a ‘conservative substitution’—e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, for example based on size, charge, polarity, hydrophobicity, chain rigidity/orientation, etc., are well known in the art of protein engineering. Polypeptides comprising conservative amino acid substitutions can be tested in any one of the assays described herein to confirm that a desired activity, e.g. binding, specificity, and/or function of a native or reference polypeptide is achieved.

The terms “dosage” or “dose” as used herein denote any form of the active ingredient formulation that contains an amount sufficient to produce a therapeutic effect with a single administration.

The term “effective amount” refers to an amount of a compound of the invention or other active ingredient sufficient to provide a therapeutic or prophylactic benefit in the treatment or prevention of a disease or to delay or minimize symptoms associated with a disease. Further, a therapeutically effective amount with respect to a compound of the invention means that amount of therapeutic agent alone, or in combination with other therapies, that provides a therapeutic benefit in the treatment or prevention of a disease. Used in connection with a compound of the present disclosure, the term can encompass an amount that improves overall therapy, reduces or avoids symptoms or causes of disease, or enhances the therapeutic efficacy or synergies with another therapeutic agent.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The term “mammal” includes, but is not limited to, humans, mice, rats, guinea pigs, monkeys, dogs, cats, horses, cows, pigs, and sheep.

A “patient” or subject” includes an animal, such as a human, cow, horse, sheep, lamb, pig, chicken, turkey, quail, cat, dog, mouse, rat, rabbit or guinea pig. The animal can be a mammal such as a non-primate and a primate (e.g., monkey and human). In one embodiment, a patient is a human, such as a human infant, child, adolescent or adult.

In this description, a “pharmaceutically acceptable salt” is a pharmaceutically acceptable, organic or inorganic acid or base salt of a compound of the invention. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The term “prevention” as used herein means the avoidance of the occurrence or of the re-occurrence of a disease as specified herein, by the administration of an active compound, for example the disclosed peptide molecules, according to the invention to a subject in need thereof.

As used herein, the terms “protein” and “polypeptide” may be used interchangeably to designate a series of amino acid residues connected to each other by peptide bonds between the alpha-amino and carboxy groups of adjacent residues. The terms “protein” and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” may be used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

“Subject in need”, “patient” or those “in need of treatment” include those already with existing disease (i.e., cancer, oncologic pathologies, breast cancer, lung cancer, head and neck cancer, etc.), as well as those at risk of the disease. The terms also include human and other mammalian subjects that receive either prophylactic or therapeutic treatments as disclosed herein.

The term “specifically binds” is “antigen specific,” is “specific for,” “selective binding agent,” “specific binding agent,” “binding affinity” or is “selective” for a target refers to a peptide molecule (e.g., CCR4-IT) that binds a target protein (e.g., CCR4) with greater affinity than other proteins, especially related proteins. It is contemplated herein that, in some embodiments, the disclosed proteins bind specifically to Treg cells, at or near the CCR4 receptor.

As used herein, the term “about” can mean relative to the recited value, e.g., amount, dose, temperature, time, percentage, etc., ±10%, ±9%, ±8%, ±7%, ±6%, ±5%, ±4%, ±3%, ±2%, or ±1%.

The singular “a,” “an,” and “the” include plural referents unless context clearly indicates otherwise. Similarly, the word “or” is intended to include “and” unless the context clearly indicates otherwise. The term “comprises” means “includes” or “contains.” Also, “comprising A or B” means including A or B, or A and B, unless the context clearly indicates otherwise. It is to be further understood that all molecular weight or molecular mass values given for compounds are approximate, and are provided for description. Although methods and materials similar or equivalent to those described herein can be used in the practice or testing of this disclosure, suitable methods and materials are described below. In addition, the materials, methods, and examples are illustrative only and not intended to be limiting.

In the following sections, certain exemplary compositions and methods are described in order to detail certain embodiments of the invention. It will be understood by one skilled in the art that practicing the certain embodiments does not require the employment of all or even some of the specific details outlined herein, but rather that concentrations, times and other specific details can be modified through routine experimentation. In some cases, well known methods or components have not been included in the description.

### DETAILED DESCRIPTION

The immunosuppressive tumor microenvironment (“TME”) is a significant barrier for the anti-tumor immune response. Immunosuppressive cells including regulatory T cells (“Treg cells”) are often infiltrated in the immunosuppressive TME. The methods and compositions disclosed herein provide an effective cancer immunotherapy that may involve effectively targeting and depleting Treg cells.

The disclosed methods of cancer treatment may involve administering to a subject afflicted with cancer an engineered immunotoxin configured to target and deplete CCR4+ Treg cells, e.g., CCR4+ tumor-infiltrating effector Treg cells. The immunotoxin may be a genetically engineered, truncated diphtheria toxin-based recombinant single-chain fold-back diabody anti-human CCR4 immunotoxin (“CCR4-IT”). A pharmaceutical composition containing CCR4-IT may be administered alone or together with one or more additional pharmaceutical compositions or agents, including, for example, those comprising a monoclonal antibody configured to target and deplete the programmed death-1 protein (“PD-1”). Administration of CCR4-IT alone or together with an anti-PD-1 antibody may cause a subject's immune system to fight the cancer with which the subject is diagnosed. This immunotherapeutic effect may be employed by the approaches disclosed herein to combat a variety of cancer types, which may be exacerbated or associated with an immunosuppressive TME.

The disclosed compositions and associated methods of administration may therefore effectively treat a broad spectrum of cancer types, including but not limited to breast cancer, T-cell acute lymphoblastic leukemia (“T-ALL”), cutaneous T-cell lymphoma (“CTCL”), lung cancer, and/or head and neck cancer. Certain non-limiting examples may involve the treatment of triple-negative breast cancer, specifically.

While not being bound to any particular theory, administering one or more of the disclosed pharmaceutical compositions to a subject afflicted with cancer may significantly reduce, minimize, or deplete CCR4+ tumor-infiltrating effector Treg cells, which may otherwise contribute or exacerbate a variety of cancers. The mechanism of CCR4-IT may differ from that of corresponding monoclonal antibodies, the therapeutic effect of which often relies on accessory cells from the innate immune system to initiate antibody-dependent cellular cytotoxicity (ADCC), complement-dependent cytotoxicity (CDC), or antibody-dependent cellular phagocytosis (ADCP). Heavily pre-treated cancer patients often have poor accessory cell function and can therefore easily develop resistance to therapeutic antibodies. In contrast, the disclosed immunotoxin, CCR4-IT, does not rely on accessory cell function and therefore may avoid resistance mechanisms attributed to poor accessory cell function. CCR4 may therefore mediate efficient depletion within tissues including lymph node and skin.

Effective cancer treatment achieved by implementing embodiments of the present disclosure may be evidenced by reductions in tumor size (including mass and dimensions), tumor number, tumor metastasis, and/or tumor growth, along with a reduction in the number and/or proliferation of cancer cells in a subject. Effective treatment may also comprise a boost in anti-tumor immunity and/or the prolonged survival of subjects afflicted with cancer. One or more of these effects may be accompanied by a reduction or elimination of one or more symptoms associated with cancer.

### CCR4 Immunotoxin

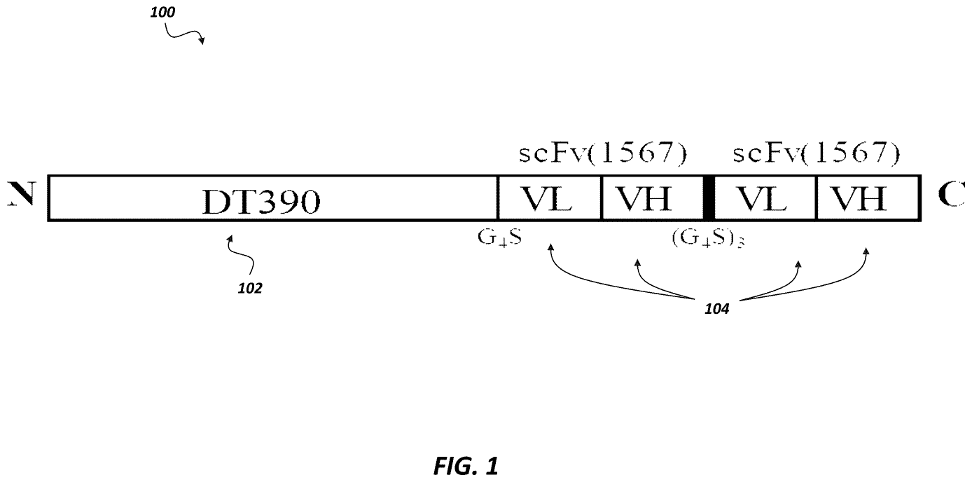

Embodiments of the engineered CCR4 immunotoxin disclosed herein may include a genetically engineered, truncated diphtheria toxin-based recombinant single-chain fold-back diabody anti-human CCR4 immunotoxin. FIG. 1 is a schematic diagram of CCR4-IT. As shown, CCR4-IT 100 may include a truncated diphtheria toxin 102 fused to two anti-human CCR4 single-chain variable fragments (“scFv”) 104. The specific toxin may be truncated diphtheria toxin DT390 in some examples.

Altogether, CCR4-IT may be about 96.31 kDa in some embodiments. Additional variations of the recombinant protein may vary in size, ranging from about 90 kDa to about 110 kDa, or any value therebetween, including about 91 kDa, about 92 kDa, about 93 kDa, about 94 kDa, about 95 kDa, about 96 kDa, about 97 kDa, about 98 kDa, about 99 kDa, about 100 kDa, about 101 kDa, about 102 kDa, about 103 kDa, about 104 kDa, about 105 kDa, about 106 kDa, about 107 kDa, about 108 kDa, about 109 kDa, about 110 kDa.

CCR4-IT was developed using a novel, advanced diphtheria toxin-resistant yeast (Pichia pastoris) expression system. The yeast expression system overcame expression and purification problems encountered using E. coli-based expression systems to deliver high production level and excellent purification quality of CCR4-IT.

A CCR4-IT peptide may have an amino acid sequence at least about 90%, 95%, or 100% identical to SEQ ID NO: 1. Administration of a CCR4-IT recombinant protein similar or identical to SEQ ID NO: 1 may be effective to treat one or more cancers and associated symptoms, as disclosed herein.

### Pharmaceutical Compositions

The pharmaceutical compositions of this disclosure are suitable for treating a variety of cancers, e.g., breast cancer. Embodiments may be especially effective in treating cancerous conditions driven or exacerbated by the presence of CCR4+ Treg cells, which may be present or concentrated in a tumor microenvironment. Embodiments of the pharmaceutical composition may include at least one CCR4-IT recombinant protein disclosed herein, which may be similar or identical to the CCR4-IT shown schematically in FIG. 1. The pharmaceutical composition may also include a pharmaceutically acceptable carrier configured to facilitate and/or stabilize CCR4-IT during and beyond delivery to the target site(s) of a subject.

In some examples, a pharmaceutical composition administered to a subject may additionally or alternatively include a therapeutically effective amount of an anti-PD-1 monoclonal antibody, such as Pembrolizumab. Such a pharmaceutical composition may include a pharmaceutically acceptable carrier configured to facilitate and/or stabilize the antibody during and beyond delivery to the target site(s) of a subject. Examples may involve co-administering one pharmaceutical composition containing CCR4-IT and another pharmaceutical composition containing the anti-PD-1 antibody.

In embodiments, the pharmaceutical composition(s) may include or be administered concurrently with one or more excipients. Suitable excipients may vary depending upon the particular dosage form utilized. In addition, suitable excipients may be chosen for a particular function, such as the ability to facilitate the production of stable dosage forms. Excipients may also be chosen for regulatory compliance. Non-limiting excipient examples include: fillers, binders, disintegrants, lubricants, glidants, granulating agents, coating agents, wetting agents, solvents, co-solvents, suspending agents, emulsifiers, coloring agents, anticaking agents, humectants, chelating agents, plasticizers, viscosity agents, antioxidants, preservatives, stabilizers, and surfactants. In some embodiments, one or more delivery vehicles may be utilized, non-limiting examples of which may include nanoparticles, nanogels, and/or adeno-associated viral vectors (“AAV vectors”). The skilled artisan will appreciate that certain pharmaceutically acceptable excipients may serve more than one function and may serve alternative functions depending on how much of the excipient is present in the final composition and which other ingredients are present in the composition.

In embodiments, the CCR4-IT protein (and anti-PD-1 antibody) may be administered concurrently with one or more buffering agents and/or diluents, non-limiting examples of which may include various concentrations of sodium hydroxide and sodium phosphate.

### Therapeutic Approaches

Methods of treating a cancer may involve administering to a subject a therapeutically effective amount of a pharmaceutical composition containing CCR4-IT. The composition may be administered after a cancer is diagnosed, for example after performing a biopsy. In some examples, prophylactic administration may be performed after determining that a subject has a tumor or signs thereof. Administration of a disclosed pharmaceutical composition may be uniquely effective at targeting and reducing the number of Treg cells in a subject, including CCR4+ Treg cells in a tumor microenvironment. Such targeted reduction may be sufficient to prevent, slow, and/or reverse further proliferation and metastasis of one or more cancerous tumors and/or cancerous cells. Accordingly, administering CCR4-IT in the manner disclosed herein may provide an effective, versatile cancer immunotherapy.

A CCR4-IT pharmaceutical composition may be infused or injected into a subject soon after the subject is diagnosed with cancer. A broad spectrum of cancer types may be effectively treated via administration of CCR4-IT, non-limiting examples of which may include breast cancer, triple-negative breast cancer, CTCL, T-ALL, lung cancer, head and neck cancer, or combinations thereof.

The frequency of administration of the pharmaceutical compositions disclosed herein may vary. In embodiments, a pharmaceutically effective amount of the composition may be administered daily, weekly, monthly, or yearly. The number of times the disclosed compositions are administered to a subject, along with the length of the treatment period, may depend on the severity or type of the cancerous condition. The length of the treatment period may also be patient-specific and re-evaluated periodically by a physician or other health care provider. In various embodiments, a pharmaceutical composition may be administered immediately following a cancer diagnosis, such as within one, two, six, 12, or 24 hours, or within one, two, three, four, five, six, seven days or more, including one or more weeks or months. The formulations may be administered once or multiple times, for example two, three, four, five, six, seven, eight, nine, ten times, or more. The pharmaceutical composition(s) may be administered to a subject for a predefined period of time, or until the condition is effectively treated, for example when all tumors have been depleted and/or removed, or when cancer cell proliferation has stopped or reversed.

As noted in the preceding section, methods of treatment may also involve administering to a subject a therapeutically effective amount of a pharmaceutical composition containing an antibody configured to target the PD-1 protein. A non-limiting example of such a composition may comprise Pembrolizumab, and it may be co-administered alongside CCR4-IT to a subject afflicted with breast cancer, e.g., triple-negative breast cancer. Co-administration of the pharmaceutical compositions may involve administering the compositions simultaneously, sequentially, or intermittently. The compositions may be administered at the same or different frequency. For example, a CCR4-IT pharmaceutical composition may be administered to a subject more frequently than an anti-PD-1 pharmaceutical composition. In various non-limiting embodiments, a CCR4-IT pharmaceutical composition may be administered daily, while the anti-PD-1 pharmaceutical composition may also be administered daily, or once every other day, once every three days, once every four days, once every five days, once every 6 days, once every week, once every two weeks, once every three weeks, once every month, etc. Co-administration of pharmaceutical compositions containing CCR4-IT and PD-1, together or separately, may result in a synergistic therapeutic effect, such that treatment of the subject afflicted with cancer is more effective than when either pharmaceutical composition is administered alone. Therapeutic synergy may be evidenced by more significant reductions in tumor size, metastasis, and/or growth, along with resulting increases in subject survival and quality of life.

Embodiments of each pharmaceutical composition may include, or be administered concurrently with, at least one pharmaceutically acceptable carrier. Relatedly, the pharmaceutical composition may be administered singly or in combination with other therapeutic agents, either serially or simultaneously. Such additional agents may or may not be formulated to treat the same conditions.

The pharmaceutical compositions disclosed herein may be administered using an infusion system or device, or an injection device, such as tuberculin syringe or an IV drip device, which may be configured specifically for the purposes described herein. In some examples, the administration device may be a single-use device, which may be included in a kit that also includes a single dose of a pharmaceutical composition. Accordingly, an injection device may constitute a part of a system for treating, reducing the risk of, preventing, or alleviating at least one symptom of retinal damage.

The therapeutically effective amount of a pharmaceutical composition administered to a subject may vary. In embodiments, a disclosed pharmaceutical composition containing CCR4-IT may be administered in a variety of doses ranging from about 5 μg/kg to about 50 μg/kg, about 10 μg/kg to about 40 μg/kg, about 15 μg/kg to about 30 μg/kg, about 20 μg/kg to about 25 μg/kg, or any level therebetween, including for example about 10 μg/kg, 12 μg/kg, 14 μg/kg, 16 μg/kg, 18 μg/kg, 20 μg/kg, 22 μg/kg, 24 μg/kg, 26 μg/kg, 28 μg/kg, or about 30 μg/kg. In some examples, the dose may not exceed about 30 μg/kg to avoid or minimize undesired off-target effects caused by CCR4-IT. Dosing may depend, for example, on the condition treated, the severity of the condition, the nature of the formulation (e.g., with or without a second pharmaceutical agent, e.g., an anti-PD-1 antibody), the method of administration, the condition of the subject, the age of the subject, the weight of the subject, or combinations thereof. Dosage levels are typically sufficient to achieve a concentration at the site of action that is at least the same as a concentration that has been shown to be active in vitro, in vivo, or in tissue culture.

The therapeutically effective amount of a pharmaceutical composition containing anti-PD-1 antibody may also vary, and may be substantially the same or different than the therapeutically effective amount of a pharmaceutical composition containing CCR4-IT. In some examples, the anti-PD-1 dose may be greater than the CCR4-IT dose, e.g., about 5 mg/kg, or ranging from about 1 mg/kg to about 10 mg/kg, including about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, or more, or any concentration therebetween.

To accommodate multiple administration techniques and schedules, the pharmaceutical compositions disclosed herein may be prepared in a unit-dosage form or multiple-dosage form, along with a pharmaceutically acceptable carrier and/or excipient according to a method employed by those skilled in the art. Example formulations may be in the form of an aqueous or oil-based solution, a suspension, or an emulsion. For increased stability and long-term storage, the pharmaceutical compositions may be lyophilized.

### Kits

This disclosure further relates to a kit comprising a pharmaceutical composition (or compositions) of this disclosure for use in a method of treating or alleviating a symptom of a cancer. In certain embodiments, kits are provided for storage, transport and use in treating or alleviating a target disease, such as a cancer, as described herein. In some embodiments, kits can include one or more containers.

### EXAMPLES

The following examples are included to illustrate certain embodiments. It should be appreciated by those of skill in the art that the techniques disclosed in the examples represent techniques discovered to function well in the practice of the claimed methods, compositions and systems. However, those of skill in the art should, in light of the present disclosure, appreciate that changes can be made in some embodiments which are disclosed and still obtain a like or similar result without departing from the spirit and scope of embodiments of the inventions.

### EXAMPLES

### Example 1

In a first example, the disclosed fold-back diabody CCR4-IT was evaluated in vivo for its efficacy in treating cancer via targeted therapy in an immunodeficient mouse tumor model. Efficacy was first assessed using a CCR4+CD25+ CTCL Hut102/6TG tumor-bearing immunodeficient NSG mouse model. Tumor cells were intravenously injected into the test mice on day 0 of the experiment. The immunotoxins were intraperitoneally injected for 10 consecutive days, starting at day 4 after tumor cell injection.

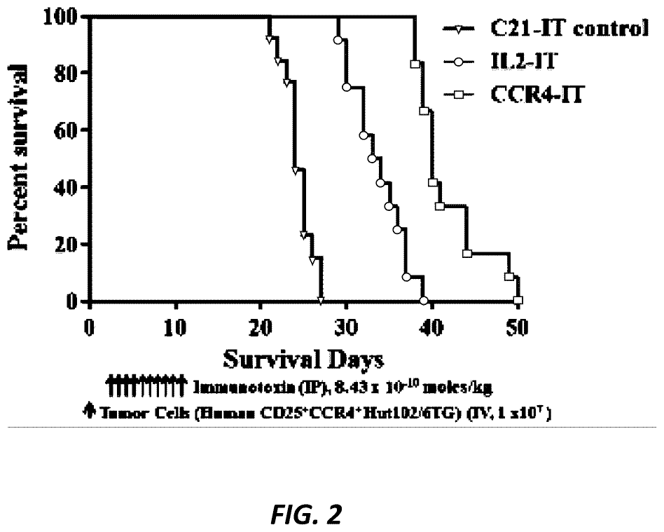

As shown in FIG. 2, CCR4-IT administration significantly prolonged the median survival of the CCR4+CD25+ CTCL tumor-bearing NSG mice relative to the negative control mice administered C21-IL2 (a non-related diphtheria toxin-based immunotoxin), extending the median survival from 24 days (21-27; n=13) to 40 days (38-50)(n=14). This median survival of the CCR4-IT group was also significantly longer than the median survival of 33.5 days (29-39) observed in a separate test group of mice administered Ontak®-like IL2-IT (n=12).

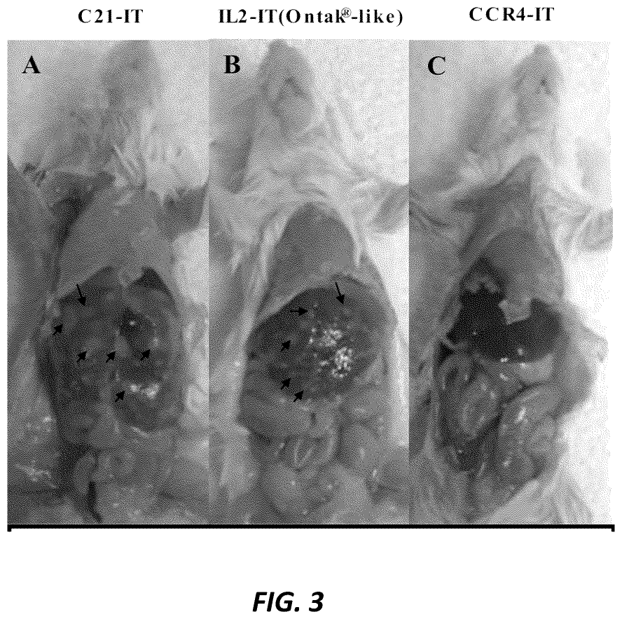

Gross examination during necropsy on day 21 revealed enlarged livers with extensive CTCL tumor nodules on the liver surface of the negative control C21-IT group. As further shown in FIG. 3, the Ontak®-like IL2-IT group had less hepatomegaly with fewer tumor nodules when compared with the C21-IT control group. The CCR4-IT group, however, demonstrated normal liver size without tumor nodules, demonstrating superior efficacy over the Ontak®-like 1L2-IT.

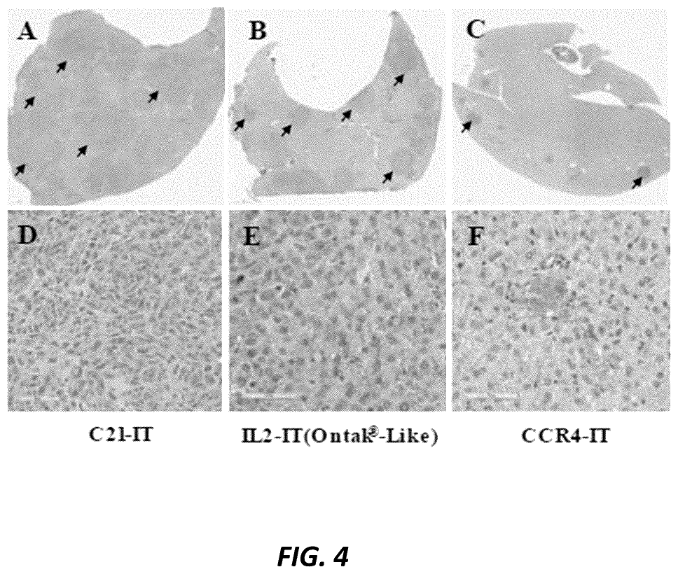

Histological evaluation of the liver was performed at day 21 for three representative tumor-bearing NSG mice. For the negative control C21-IT group, hematoxylin and eosin staining revealed replacement of liver parenchyma with extensive tumor nodules (FIG. 4). In the Ontak®-like IL2-IT group, fewer tumor cell areas were seen in the examined section of the liver compared to that of the negative control C21-IT group (FIG. 4). Pathology of the CCR4-IT group showed only sporadic tumor cell areas, demonstrating that CCR4-IT treatment was significantly more effective against the tumor cells than Ontak®-like IL2-IT and C21-IT treatment. Taken together, in vivo efficacy data consistently demonstrated that the CCR4-IT effectively treated mice afflicted with CCR4+CD25+ CTCL relative to the negative control and Ontak®-like IL2-IT mice. Accordingly, administration of the disclosed fold-back CCR4-IT may effectively treat CTCL.

### Example 2

The in vivo efficacy of the disclosed fold-back diabody CCR4-IT to effect targeted therapy in a CCR4+ T-ALL-bearing immunodeficient mouse tumor model was also assessed. A C21-IT negative control group was again used for comparison, as was a test group administered a monovalent CCR4-IT, and a test group administered bivalent CCR4-IT. NSG mice were IV injected with 1.0×10⁷ CCR4+ CCRG=CEM leukemia cells and treated from day 0 on with the test immunotoxins at 50 μg/kg BID for four consecutive days as one course, two course total, and three-day break between the two courses.

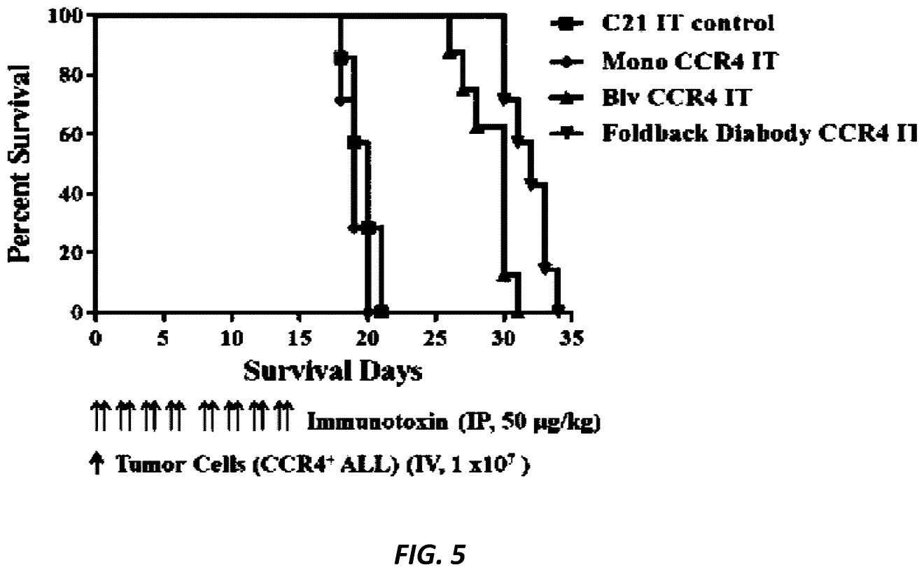

As shown in FIG. 5, the fold-back diabody CCR4-IT effectively depleted CCR4+ T-ALL, evidenced by a significantly prolonging of the survival of the CCR4+ T-ALL tumor-bearing mice compared with the negative control group (median survival days 32 vs 20). The monovalent anti-human CCR4 immunotoxin group (mono CCR4 IT, n=7) had a median survival time of only 19 days. The bivalent anti-human CCR4 immunotoxin group (Biv CCR4-IT, n=8) had a median survival time of 30 days. The single chain fold-back diabody anti-human CCR4 immunotoxin group (fold-back diabody CCR4-IT, n=7) had a median survival time of 32 days. Accordingly, administration of the disclosed fold-back CCR4-IT may effectively treat T-ALL via targeted therapy.

### Example 3

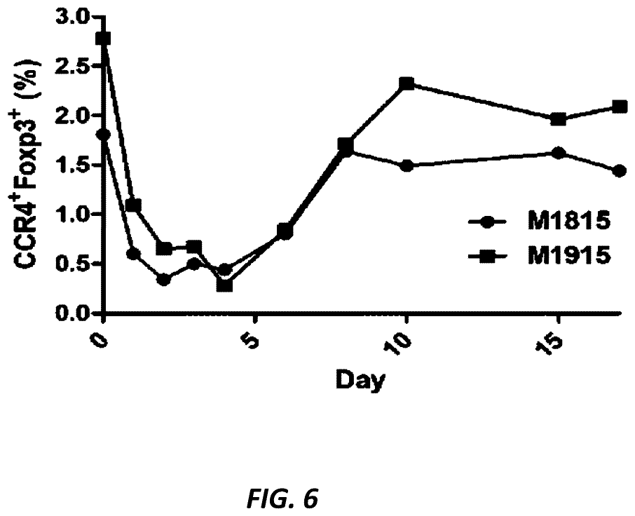

The efficacy of the disclosed fold-back CCR4-IT to deplete CCR4+Foxp3+ Treg cells in both naïve cynomolgus monkeys and minipigs was also evaluated. The immunotoxin was injected at 25 μg/kg, IV, BID for four consecutive days. M1815 and M1915 represent two cynomolgus monkeys. The CCR4+Foxp3+ Treg depletion in the peripheral blood was monitored by flow cytometry using antibodies against human CCR4 and Foxp3 (CCR4+Foxp3+ among the gated CD4+cells). As shown in FIG. 6, 78-89% monkey CCR4+Foxp3+ Treg cells were effectively depleted in the peripheral blood lasting approximately for 10 days, and 89-96% monkey CCR4+Foxp3+ Treg cells were also effectively depleted in the lymph nodes. No effect was observed in other monkey cell populations including CD8+ T cells, other CD4+ T cells, B cells and NK cells. Accordingly, the disclosed fold-back CCR4-IT effectively depleted non-human primate Treg cells, which may otherwise contribute to a variety of cancers.

### Example 4

The in vivo efficacy of the disclosed fold-back CCR4-IT for breast cancer immunotherapy via depletion of tumor-infiltrating effector Treg cells was assessed using a humanized CDX mouse tumor model.

Human triple-negative breast cancer (TNBC) MDA-MB-231 cells (ATCCHTB-26) were used to establish the solid humanized mouse tumor model. Ten million of the MD-MAB-231 tumor cells were subcutaneously (SQ) injected into mammary fat pads on day 0. Fold-back diabody CCR4-IT was intraperitoneally (IP) injected from day 4 at 50 μg/kg, and once daily for 10 consecutive days. C21 immunotoxin (C21-IT, 50 μg/kg) was included as the negative control. Altogether, six mice were included in each treatment group, which included a CCR4-IT test group administered the fold-back CCR4-IT disclosed herein, a CCR4-IL2-IT test group, an IL2-IT test group, and a negative control group administered C21-IT. The mice were administered a molar dose of 8.43×10¹⁰ moles/kg. Tumors were measured by caliper every other day and the tumor volumes (mm³) were calculated by (length×width)2/2. Blood samples from tail vein before treatment and after the treatment were collected for immunophenotyping analysis (human CD45, CD3, CD4, CD8, and Foxp3).

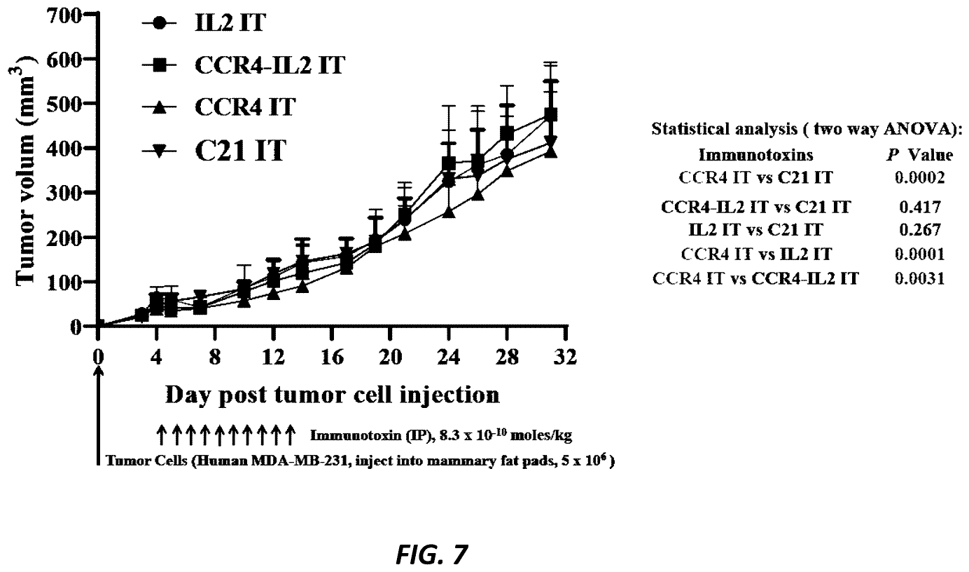

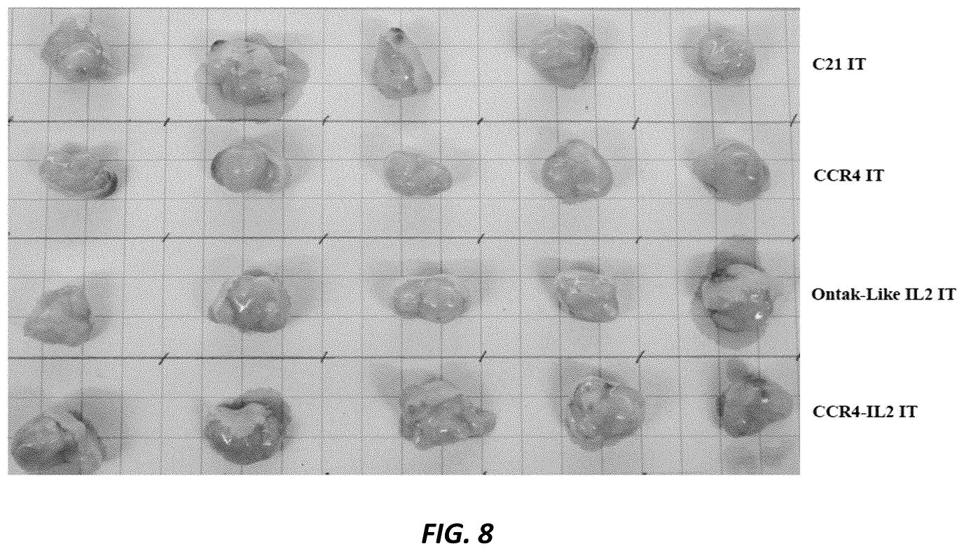

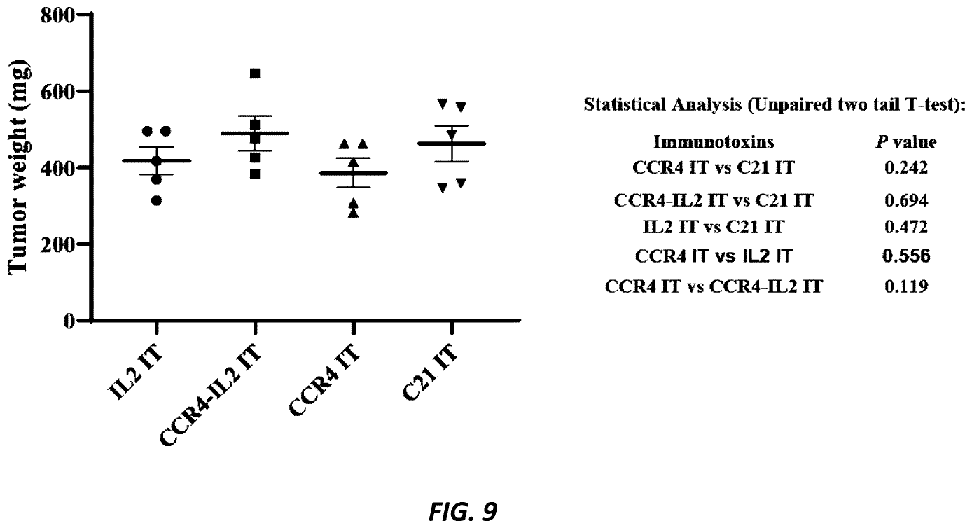

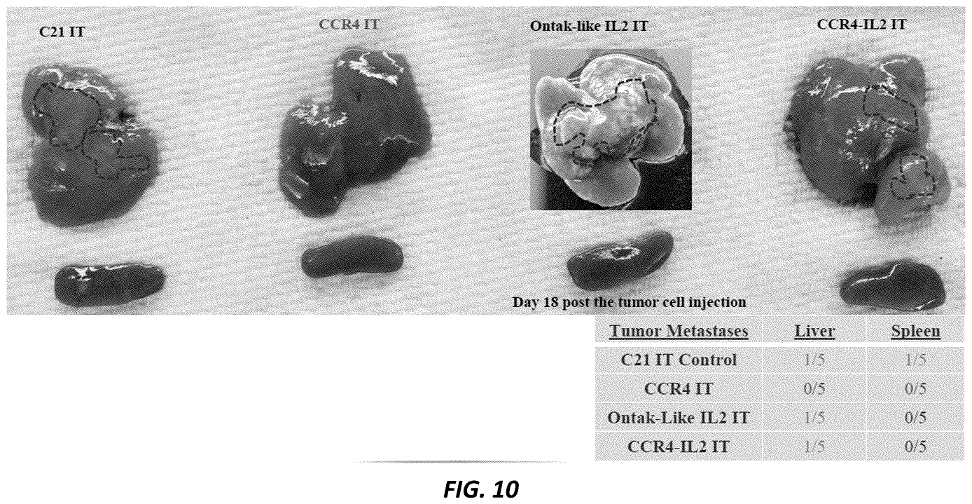

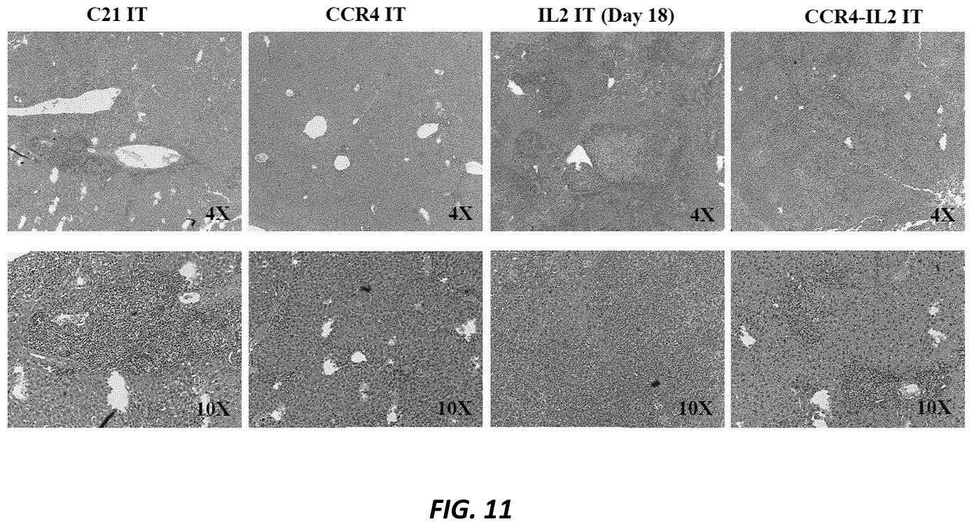

As shown in FIG. 7, tumors extracted from the mice administered CCR4-IT had the smallest average tumor volume, indicating that CCR4-IT may decrease tumor volume and potentially slow tumor growth relative to other treatments in subjects having breast cancer. Images of representative tumors extracted from the mice in each group are shown in FIG. 8. Tumors extracted from the CCR4-IT mice were visibly smaller than tumors extracted from the other mice, consistent with the numerical measurements represented in FIG. 7. The tumors were also weighed, and as shown in FIG. 9, tumors extracted from the CCR4-IT mice weighed the least on average. Tumor metastasis was also reduced in the CCR4-IT mice relative to the others, as shown in FIG. 10. In particular, the CCR4-IT mice were the only mice in which tumors did not metastasize to the liver or spleen during the course of the experiment. Immunostaining further confirmed these results, as shown in FIG. 11.

### Example 5

In a separate experiment, the efficacy of the disclosed fold-back CCR4-IT for breast cancer immunotherapy, either alone or in combination with an anti-PD-1 antibody (“Pembrolizumab”) was evaluated.

Human triple-negative breast cancer (TNBC) MDA-MB-231 cells (ATCCHTB-26) were used to establish the solid humanized mouse tumor model. Ten million of the MD-MAB-231 tumor cells were subcutaneously (SQ) injected into mammary fat pads on day 0. Fold-back diabody CCR4-IT was intraperitoneally (IP) injected starting from day 4 at 81.2 μg/kg, and once daily for 10 consecutive days, in the CCR4-IT test group. Altogether, six mice were included in each treatment group, which included the CCR4-IT test group administered the fold-back CCR4-IT disclosed herein. A group administered 5 mg/kg/day of Pembrolizumab was included, as was a combination test group administered both CCR4-IT and Pembrolizumab (81.2 μg/kg and 5 mg/kg, respectively), along with a negative control group administered C21-IT (59 μg/kg/day). Pembrolizumab was administered intraperitoneally on days 4 and 9 of the experiment, after tumor cell IV injection.

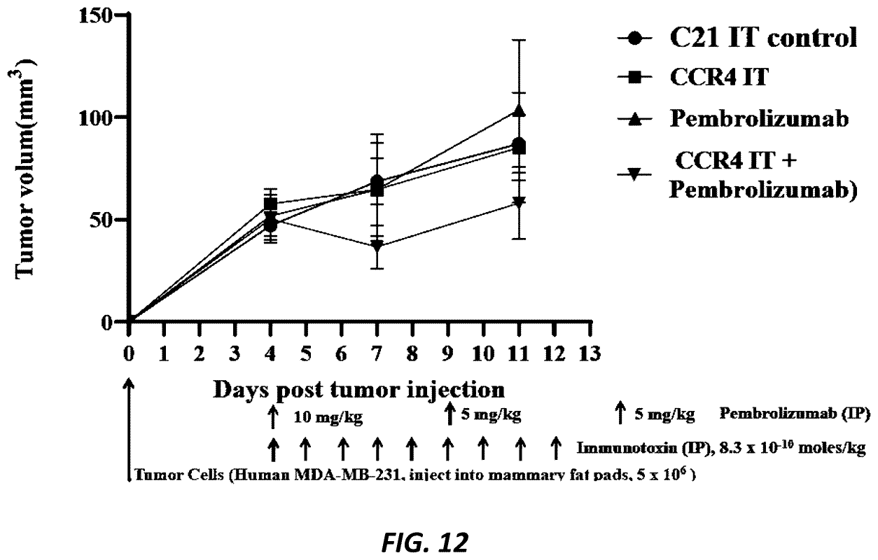

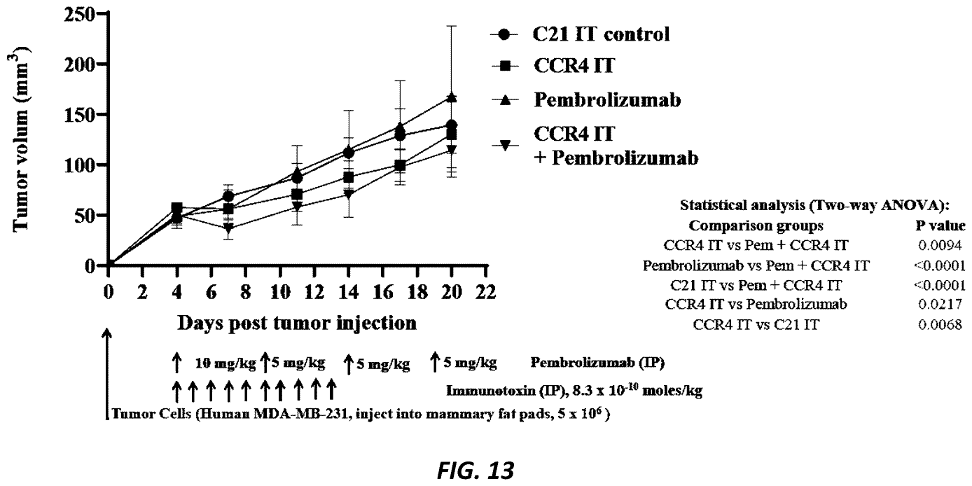

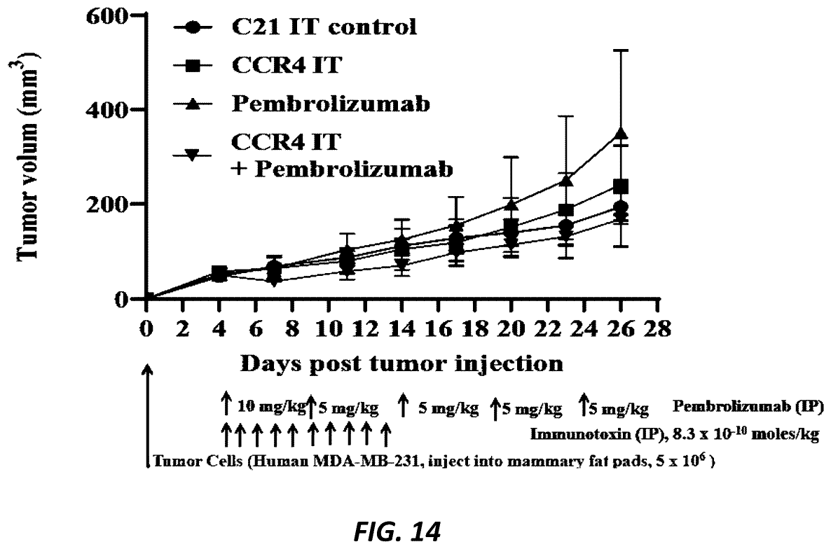

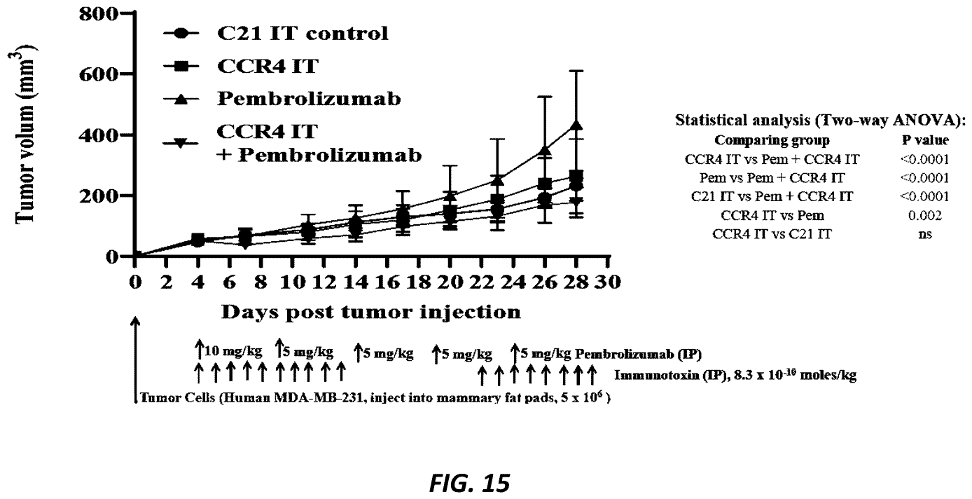

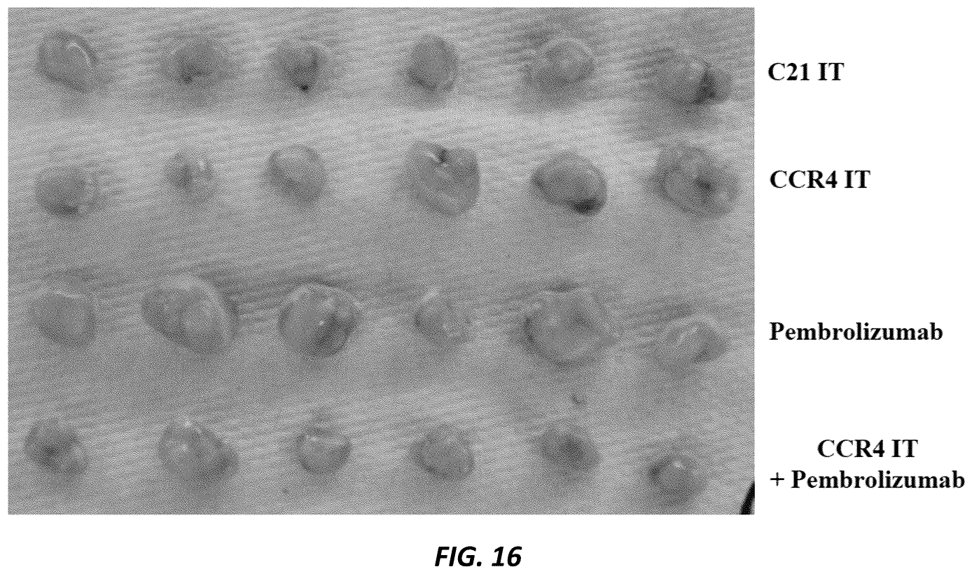

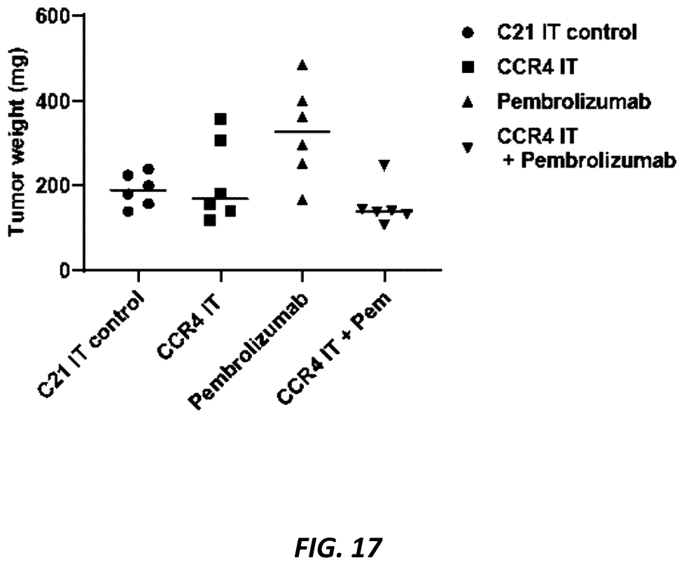

As shown in FIG. 12, the average volume of tumors extracted from the mice administered both CCR4-IT and Pembrolizumab was the smallest, followed by the CCR4-IT group, 13 days after tumor cell injection. FIG. 13 shows that after 22 days, tumor volume remained the smallest for the CCR4-IT+Pembrolizumab group. This effect persisted through day 28 (FIG. 14) and day 30 (FIGS. 15 and 16). Tumor weight was consistent with tumor volume, as the combination test group administered CCR4-IT and Pembrolizumab had the lowest average tumor weight relative to the other test groups (P value of 0.0057), shown in FIG. 17 and below in Table 1. Accordingly, the disclosed fold-back CCR4-IT and Pembrolizumab may exhibit a synergistic effect in treating breast cancer when administered together to a subject afflicted with the disease.

TABLE 1

| | C21 IT | | | CCR4-IT + |
|---|---|---|---|---|
| Tumors | control | CCR4-IT | Pembrolizumab | Pembrolizumab |
| 1 | 224 | 154.6 | 295.4 | 140.8 |
| 2 | 200.4 | 119.3 | 483.4 | 246.9 |
| 3 | 139.1 | 141.1 | 361.2 | 107.7 |
| 4 | 157.1 | 306.2 | 167.7 | 137.9 |
| 5 | 180.2 | 182.4 | 400.5 | 144.3 |
| 6 | 239.3 | 356.7 | 251.8 | 132.3 |
| Average | 190.02 | 210.05 | 326.67 | 151.65 |

### Example 6

In this experiment, the efficacy of the disclosed fold-back CCR4-IT for breast cancer immunotherapy via Treg depletion was assessed in immunodeficient NSG mice as in vivo negative control study.

A triple-negative breast cancer cell line MDA-MB-231 was obtained from the American Type Culture Collection (ATCC). The growth medium used was ATCC-formulated Leibovitz's L-15 medium (catalog No. 30-2008), supplemented with 10% fetal bovine serum. The 15-16-week-old female Hu-CD34⁺ NSG-SGM3 (HuNSG) and NSG mice were obtained from Jackson Laboratory (Bar Harbor, Maine, USA). All animal procedures were performed in accordance with the Guide for the Care and Use of Laboratory Animals [Institute of Laboratory Animal Resources (U.S.). Committee on Care and Use of Laboratory Animals. Guide for the care and use of laboratory animals, p volumes.], which can be found in an AAALAC-accredited facility and approved by the University of Colorado Institutional Animal Care and Use Committee (AAALAC Accreditation No. 00235; Protocol 00116).

For the MDA-MB-231 tumor model, 5×10⁶ tumor cells were injected subcutaneously into the mammary fat pads of HuNSG or NSG mice. Treatment was initiated when the tumors reached a volume of 50-100 mm³. The vehicle control PBS (Corning, Mediatech Inc, VA, USA) was administered to the negative control mice. An immunotoxin control porcine CD3 immunotoxin (“pCD3-IT”) was administered intraperitoneally to one test group for 10 consecutive days at a daily dose of 0.08 mg/kg, the disclosed fold-back CCR4 immunotoxin (“CCR4-IT”) was administered intraperitoneally to another test group for 10 consecutive days at a daily dose of 0.08 mg/kg. Pembrolizumab (Anti-PD-1, MedChemExpress LLC, NJ, USA) was injected (IP) at a dose of 10 mg/kg for the first dose, followed by a dose of 5 mg/kg every 5 days until the study endpoint in two additional test groups. A combination of CCR4-IT and Pembrolizumab was administered to another test group, as was the combination of Pembrolizumab and pCD3-IT. Tumors were measured by caliper every 3-4 days, and tumor volumes (mm³) were calculated using the formula (length×width²)/2.

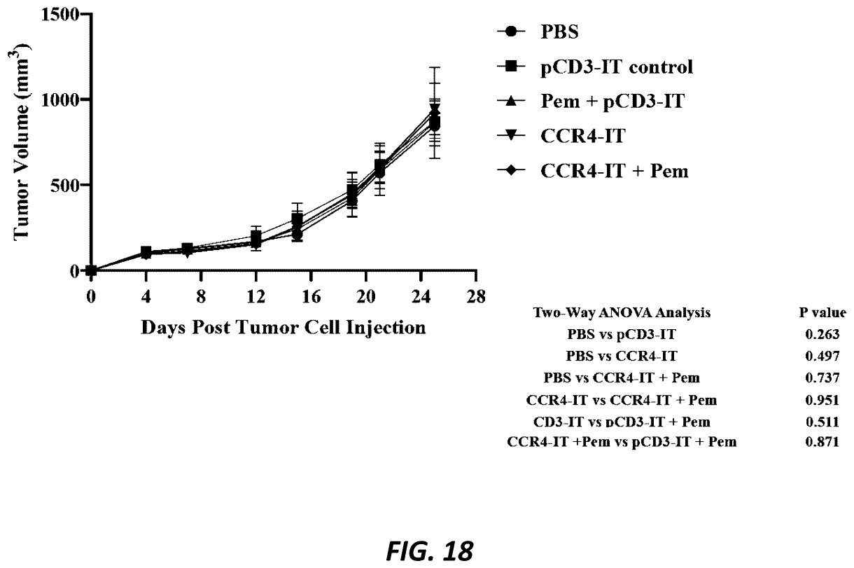

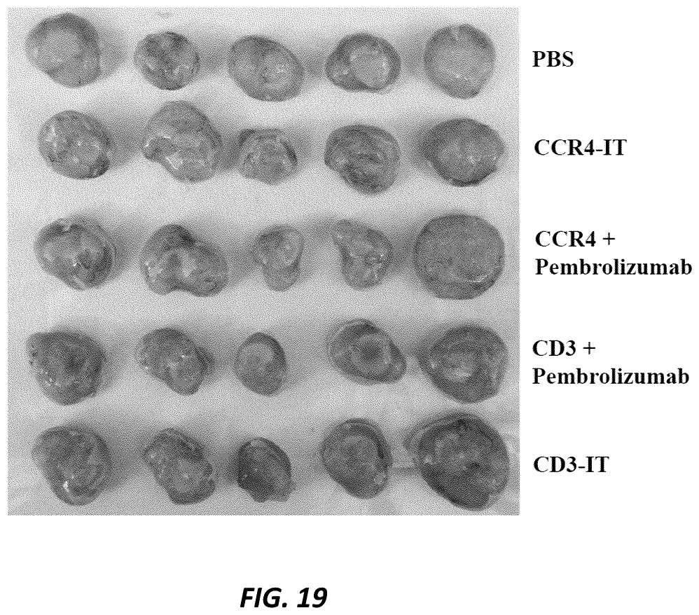

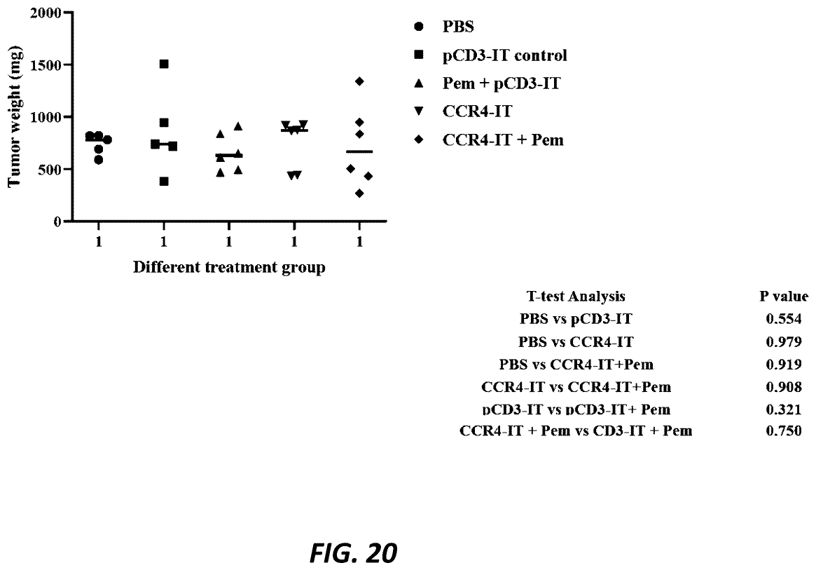

As shown in FIGS. 18-20, effective immunotherapy was not observed in immunodeficient NSG mice administered CCR4-IT, Pembrolizumab, or both. In particular, FIG. 18 shows that no significant differences in tumor volume were observed in any of the treatment groups relative to the negative PBS control group. The tumor photographs shown in FIG. 19 reiterate this finding, as do the FIG. 20 data showing no significant differences in tumor weight between any of the treatment groups and the negative control. Accordingly, in the absence of an active immune system, effective immunotherapy otherwise provided by the combination of CCR4-IT and Pembrolizumab may not be observed. This finding further evidences the efficacy of CCR4-IT and Pembrolizumab as an immunotherapeutic system.

While multiple embodiments are disclosed, still other embodiments of the present invention will become apparent to those skilled in the art from the foregoing description. As will be apparent, the invention is capable of modifications in various obvious aspects, all without departing from the spirit and scope of the present invention. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

What is claimed is:

1. A method of treating or alleviating at least one symptom of a breast cancer in a subject diagnosed with the breast cancer, comprising:
administering to the subject a therapeutically effective amount of a pharmaceutical composition comprising a genetically engineered C-C Motif Chemokine Receptor 4 (“CCR4”) immunotoxin.

2. The method of claim 1, wherein the breast cancer comprises triple-negative breast cancer.

3. The method of claim 1, wherein the CCR4 immunotoxin is a diphtheria toxin-based recombinant fold-back diabody anti-human CCR4 immunotoxin.

4. The method of claim 3, wherein the CCR4 immunotoxin comprises two tandem anti-human CCR4 scFv fused to a truncated diphtheria toxin DT390.

5. The method of claim 1, further comprising administering a pharmaceutical composition comprising an anti-programmed death-1 (“PD-1”) monoclonal antibody to the subject.

6. The method of claim 5, wherein the pharmaceutical composition comprising the anti-PD-1 monoclonal antibody is administered concurrently with the pharmaceutical composition comprising the genetically engineered CCR4 immunotoxin.

7. The method of claim 5, wherein the pharmaceutical composition comprising the anti-PD-1 monoclonal antibody and the pharmaceutical composition comprising the genetically engineered CCR4 immunotoxin are administered separately.

8. The method of claim 1, wherein the pharmaceutical composition further comprises an anti-PD-1 monoclonal antibody.

9. The method of claim 1, wherein the CCR4 immunotoxin reduces an amount of tumor-infiltrating Treg cells in the subject.

10. The method of claim 1, wherein the pharmaceutical composition is administered intravenously.

11. The method of claim 1, further comprising performing a biopsy on a tumor within the subject.

12. The method of claim 1, wherein administering to the subject the therapeutically effective amount of the pharmaceutical composition causes a reduction in a tumor volume, weight, number, and/or metastasis.

13. A method of treating or alleviating at least one symptom of a cancer in a subject diagnosed with the cancer, the method comprising:
administering to the subject a therapeutically effective amount of a pharmaceutical composition comprising a genetically engineered CCR4 immunotoxin,
wherein the cancer comprises breast cancer, lung cancer, or head and neck cancer.

14. The method of claim 13, wherein the breast cancer comprises triple-negative breast cancer.

15. The method of claim 13, wherein the CCR4 immunotoxin is a diphtheria toxin-based recombinant fold-back diabody anti-human CCR4 immunotoxin.

16. The method of claim 15, wherein the CCR4 immunotoxin comprises two tandem anti-human CCR4 scFv fused to a truncated diphtheria toxin DT390.

17. The method of claim 13, further comprising administering a pharmaceutical composition comprising an anti-PD-1 monoclonal antibody to the subject.

18. A system for treating or alleviating at least one symptom of a breast cancer in a subject diagnosed with the breast cancer, the system comprising:
an injection device configured to administer to the subject a therapeutically effective amount of a pharmaceutical composition comprising a genetically engineered CCR4 immunotoxin.

19. A pharmaceutical composition formulated for treating a breast cancer in a subject, the pharmaceutical composition comprising a genetically engineered CCR4 immunotoxin and one or more excipients.

20. The pharmaceutical composition of claim 19, wherein the CCR4 immunotoxin is a diphtheria toxin-based recombinant fold-back diabody anti-human CCR4 immunotoxin.
